(19) United States
(12) Patent Application Publication
(10) Pub. No.: US 2019/0117128 A1
(43) Pub. Date: Apr. 25, 2019

(54) METHOD AND DEVICE FOR ANATOMICAL ANGLE MEASUREMENT

(71) Applicant: MELOQ AB, Stockholm (SE)

(72) Inventors: Rui CHEN, Stockholm (SE); Tommy Paul HINKS, Stockholm (SE); Bjorn Olof Jonas NUTTI, Stockholm (SE); Jerker Paul SKOGBY, Stockholm (SE); David Carl ALIND, Järfälla (SE)

(21) Appl. No.: 15/559,664

(22) PCT Filed: Mar. 18, 2016
(86) PCT No.: PCT/EP2016/055972

§ 371 Date: Sep. 19, 2017

(30) Foreign Application Priority Data
Mar. 19, 2015 (SE) 1550332-9

(51) Int. Cl. A61B 5/11; A61B 5/107; A61B 5/00

(52) U.S. Cl. CPC A61B 5/1121; A61B 5/1071; A61B 2562/0219; A61B 5/749; A61B 5/6825

(57) ABSTRACT

An alignment guide for determining a human or animal body part range of motion around a joint is disclosed. The alignment guide is adapted to be placed against the body part and to receive a sensor unit. By using the sensor unit, the range of motion of a human or an animal joint in a main plane of rotation can be estimated based on a relative angle between an orientation of the sensor unit in a first measurement position and an orientation of the sensor unit in a second measurement position. A system, as well as a method using the alignment guide and sensor unit is also disclosed.

### TECHNICAL FIELD

The invention relates generally to assessment of anatomical angles. In particular the invention relates to devices, methods and systems for measuring anatomical range of motion of a human or animal body part around a joint.

### BACKGROUND

In many instances it is required or desired to measure the range of motion of anatomical joints in humans or animals as different anatomical angles of body parts. A wide range of such, static, angles or angle ranges can be of interest. For example for many medical and rehabilitation methods the patient's ability to move different body parts are measured and logged for both assessment of flexibility, diagnosis and follow up treatment purposes. Traditionally anatomical angles have been measured with different kinds of instruments. Typically a goniometer can be used. The goniometer is for example used to document initial and subsequent range of motion during e.g. rehabilitation. It can be used to determine the extent of a permanent disability. It can also be used to evaluate effect of different treatments, rehabilitation programs etc. In another exemplary application the goniometer is used to measure range of motion in joints of the body. This measurement can be helpful for providing an objective measure of progress in a rehabilitation program. For example if a patient has a decreased range of motion in some part of the body, a therapist can use a goniometer to assess the progress during rehabilitation. Objective measurement of progress may also play an important role for patient motivation, since patient compliance in rehabilitation in many cases is a challenge.

Some efforts have been made to replace the traditional goniometer with sensors. For example US20080281235 describes a system for monitoring joint position following introduction of a joint prosthesis in a patient. The system includes a first angular movement sensor positioned adjacent a first side of a bodily joint of a patient and a second angular movement sensor positioned adjacent a second, opposite side of the bodily joint. A receiver can receive data from the angular movement sensors.

Also, the thesis “Development of Wearable Sensors for Body Joint Angle Measurement” by Saba Bakhshi Khayani describes three different methods for body joint angle measurement using wearable sensors.

There is a constant desire to improve the efficiency, accuracy, robustness of sensor based body angular measurement systems and also to provide systems that are more user-friendly.

### SUMMARY

It is an object of the present invention to provide an improved device and system for measuring a human or animal body part range of motion around a joint. This object and/or other objects can be obtained with the alignment guide, system and method as set out in the appended independent claims. Advantageous embodiments are described by the dependent claims.

According to an aspect, an alignment guide is provided. The alignment guide is adapted to be placed against the body part to be measured, and to receive a sensor unit adapted to estimate, in a main plane of rotation, the range of motion of the body part as it moves around a joint. The measurement or estimation is based on a relative angle between an orientation of the sensor unit in a first measurement position and an orientation of the sensor unit in a second measurement position.

According to another aspect, a method for determining a human or animal body part range of motion is provided. The method may be performed by means of an alignment guide and a sensor unit that is attached to the alignment guide. The method comprises the steps of placing the alignment guide against the bodypart to be measured and measuring an orientation of the sensor unit in a first measurement position and in a second measurement position. Further, the range of motion in a main plane of rotation is estimated based on a relative angle between the orientation of the sensor unit in the first measurement position and in the second measurement position.

The alignment guide may e.g. have the form of a ruler, and may be suitable for being aligned with the body part. The alignment could be understood as the process of orienting the alignment guide with the body part in a repeatable and predictable manner. The alignment could e.g. be realized by placing the alignment guide on the body part such that a main direction of extension, or length extension, of the alignment guide coincides with a corresponding main direction of extension, or length extension, of the body part. The alignment guide thus allows for an improved accuracy, reliability and repeatability of the measurements, since it may guide the user to place the alignment guide (and hence the measuring unit) in a desired orientation relative the body part.

It will be appreciated that the first measurement position and the second measurement position may correspond to the same position on the body part or two different positions on the body part.

The alignment guide may be adapted to receive the sensor unit in a releasable manner, allowing the sensor unit to be attached and removed from the alignment guide. The sensor may e.g. be attached or secured to the alignment means by any releasable fastening means, such as a snap-lock, Velcro tape, clasps, clips, screws etcetera. This allows for the sensor unit/alignment guide to be replaced so as to adapt the measurement to different body parts. It might e.g. be advantageous to use a relatively small alignment guide when measuring the range of motion around a finger joint and relatively large alignment guide when measuring the range of motion around the hip or knee.

According to an embodiment, the alignment guide may during the measurement be placed at two separate positions on the body part. The positions may e.g. be located on different sides, or opposite sides, of the joint around which the range of motion should be measured. In one example, the joint may be arranged in a static or fixed position and the relative angle or orientation of the parts of the body connected by the joint determined. The relative angle may e.g. indicate the maximum or minimum angle the joint can reach. During such a measurement, the alignment guide may be placed against the body part at the first measurement position and then moved to another location on the body part, corresponding to the second measurement position. The alignment guide may e.g. be transported between the first measurement position and the second measurement position in a non-contact manner, i.e., released from the body during transportation, or in a contact manner wherein the alignment guide rests against the body during the transportation.

According to an embodiment, the first measurement position and the second measurement position may correspond to the same position on the body part. This may e.g. correspond to the alignment unit being held against a particular position on the body part while the body part is being moved around the joint.

According to an embodiment, an activating means such as a push button may be provided. The activating means may be adapted to initiate or trigger the measurement of the orientation of the sensor unit in the first measurement position and/or in the second measurement position. In one example, the measurement sequence may be as follows:
the alignment guide is placed in the first measurement position;
a measurement of the orientation of the sensor unit in the first measurement position is triggered by means of the activating means;
the alignment guide is moved to the second measurement position; and
a measurement of the orientation of the sensor unit in the second measurement position is triggered by means of the activating means.

The trigger of the measurements may e.g. be realized by the user pressing a button, tapping the sensing unit or the alignment guide, or touching a touch sensitive area. Thus, the alignment guide and/or sensor unit may be adapted to measure the orientation in discrete positions rather than continuously. However, other embodiment may provide continuous measurements as well.

According to some embodiments, the alignment guide may form an integral part of the sensor unit. According to other embodiments, the alignment guide and the sensor unit may be provided as structurally separate parts.

According to an embodiment, the alignment guide may be adapted to contact the body part in at least two distinct points of contact. This may be advantageous e.g. when aligning the device with curved body parts, such as the surface of the head, as a flat, continuous surface or line would only be contacting the body part in one point and thus be difficult to correctly align and hold firmly against the body part.

According to an embodiment, at least one of the alignment guide and the sensor unit may be provided with a gripping means. The gripping means may be adapted to be gripped by a hand of the user, and preferably between a pair of fingers such as e.g. the index finger and the middle finger, the middle finger and the ring finger, or the ring finger and the little finger. The gripping means may e.g. be formed such that, when the alignment guide is placed against the body part, the back of the hand of the user may be facing the body part, the gripping means is gripped between two neighboring fingers. The thumb may be used e.g. for pressing an activating means being e.g. a button or switch.

Further, the gripping means may be provided with at least two pairs of substantially parallel gripping surfaces. The orientation of the alignment guide may thus be determined by the actual pair of surfaces being gripped by the user. To illustrate by an example: holding the hand in the same direction (preferably ergonomically correct for the user) and switching the grip from a first pair of surfaces to a second pair of surfaces will cause the alignment guide to change its orientation by an angle of rotation that corresponds to the angle between the first pair of surfaces and the second pair of surfaces. Thus, the alignment guide can be held against the body in a great number of orientations without the need for the user to rotate the hand to a corresponding degree.

According to an embodiment, the sensor unit may be adapted to estimate the main plane of rotation based on the sensor unit's orientation in the first measurement position and the second measurement position, respectively. Determining the main plane of rotation is advantageous in that it allows for motion in other directions or dimensions, such as twisting of the body part, to be eliminated from the measurement of the range of motion. Thus, the range of motion of e.g. a knee or elbow joint may be accurately determined even if the leg or arm at the same time would move around the hip or should joint during the measurement. This may also limit the effects from the body tissue. When a sensor unit is aligned with e.g. a limb there is a risk that the surface of the limb may not be essentially parallel to the joint plane of rotation. This tends to cause an angle difference for the sensor unit between the first measurement position and the second measurement position, which differ from the actual joint angle in the first and second measurement positions. By determining the main plane of rotation, which is an estimation of the joint plane of rotation, and calculating the angle in that plane, the error may be reduced.

According to an embodiment, the sensor unit may be adapted to estimate the main plane of rotation by finding a maximum scalar product of a set of base vector pairs of the sensor unit in the first measurement position and the second measurement position, respectively.

According to an embodiment, the sensor unit may be adapted to estimate the main plane of rotation e.g. by means of a spherical linear interpolation, SLERP, of quaternion representations of the orientation of the sensor unit in the first measurement position and the second measurement position, respectively.

In according to some examples, a system for determining body motion is provided. The system can comprise one or more sensor units. Each sensor unit may comprise an angle sensor. The system is further adapted to determine the motion of a body part to be measured based on the relative angle of two positions of a sensor or the relative angle between two sensors. The system is also adapted to, for a particular angle to be measured, determine a plane in which a predominant rotation takes place and to generate a measured angle around an axis perpendicular to the determined plane.

In accordance with one example two sensors are used and the two sensors are calibrated to a common coordination system.

In accordance with one example the angle sensor(s) comprises an IMU. The IMU can be adapted to output rotation data as a quaternion or any other angle representation. In accordance with one embodiment the system is adapted to find or approximate the direction to be measured by determining the scalar product between two base vector pairs that is the largest as the measured direction.

In accordance with one example the sensors comprises fastening means for attaching the sensors to a holder.

In accordance with one example the system comprises a holder formed as a handle.

In accordance with one example the system comprises a fastener for fastening a sensor unit to a body. The fastener can for example be implemented by straps or similar means.

In accordance with one example at least three sensors are provided.

### BRIEF DESCRIPTION OF THE DRAWINGS

Exemplary embodiments of the invention will now be described with reference to the accompanying drawings, in which:

FIG. 1 is a perspective view of an alignment guide and a sensor unit according to an embodiment of the present invention,

FIG. 2 is an example of an alignment guide and sensor unit according to an embodiment, arranged at two different measurement positions on a patient,

FIG. 3 is a perspective view of a patient wearing a system of anatomic angle measurement with two sensor units,

FIGS. 4a and 4b is a view of a sensor unit,

FIG. 5 depicts a one sensor configuration

FIG. 6 is a flowchart illustrating procedural steps performed in accordance with a first algorithm, and

FIG. 7 is a flowchart illustrating procedural steps performed in according with a second algorithm.

All the figures are schematic and generally only show parts which are necessary in order to elucidate the invention, whereas other parts may be omitted or merely suggested.

### DETAILED DESCRIPTION

Below some embodiments and examples are described. It is to be understood however, that the illustrated embodiments and examples are for illustration only and are not restrictive.

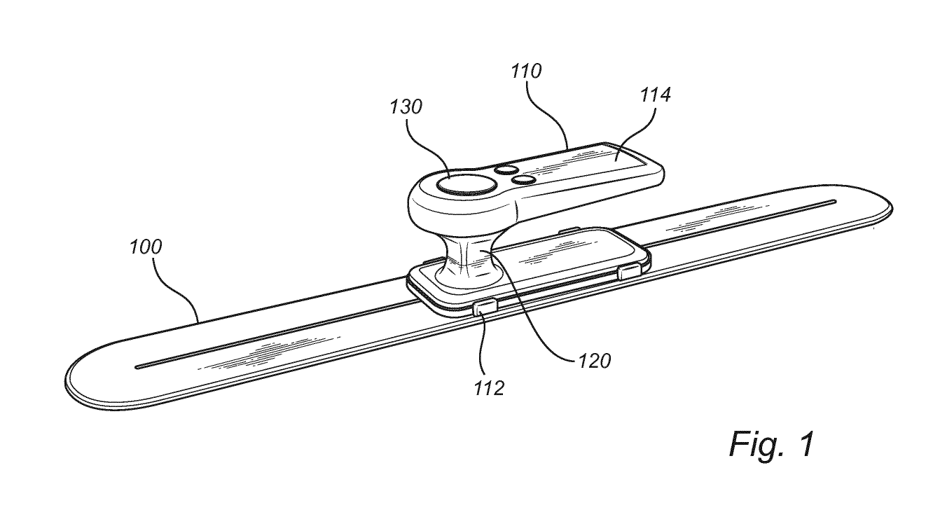

FIG. 1 shows an alignment guide 100 adapted to be positioned against the body part to be measured (not shown in FIG. 1). The alignment guide 100 shown in the present figure has a main length of extension allowing the alignment guide to be aligned with e.g. a part of an arm or a leg. Further, a sensor unit 110 is depicted, which may be releasably attached to the alignment guide 100 by means of e.g. clips or a snap-lock connection 112. The sensor unit 110 may comprise an activating means such as e.g. a push button 130, adapted to be pushed by the used so as to trigger or initiate a measurement or collection of orientation data of the sensor unit 110. A display 1 may also be provided for indicating e.g. the orientation, a measured relative angle of motion or an estimated range of motion.

Further, a gripping means 120 may be provided so as to facilitate handling and usage of the alignment guide and/or sensor 110. The gripping means 120 may be provided with a first pair of substantially parallel surface and a second pair of substantially parallel surfaces.

During operation, the alignment guide 100 and the sensor unit 110 may be gripped at the gripping means 120 and placed against, and aligned with, the body part. The relative orientation of the alignment guide 100 (or sensor unit 110) may be measured or recorded in response to the user pushing a trigger button 130.

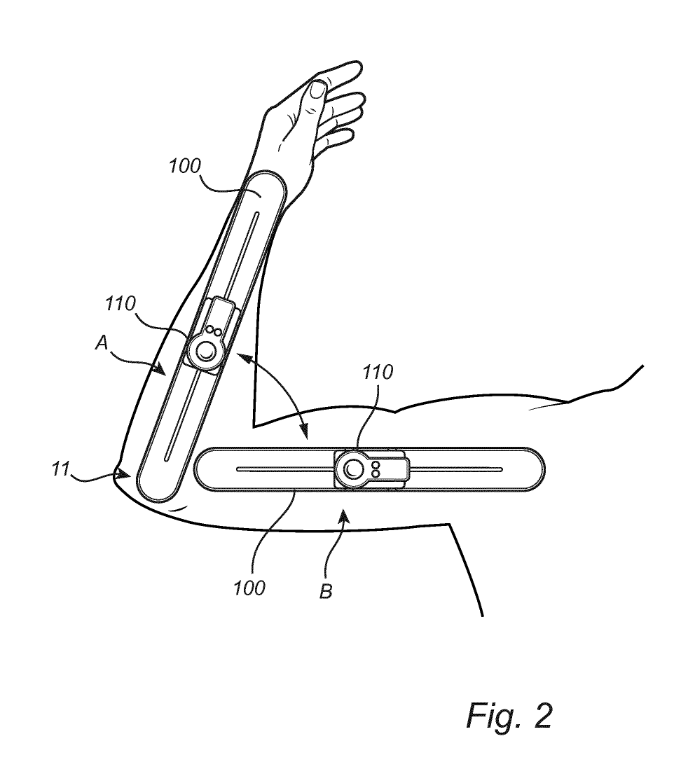

In FIG. 2, the alignment guide 100 and sensor unit 110 are illustrated when arranged at two different measurement positions A, B on a body part such as e.g. an arm. The movement of the alignment guide 100 and sensor unit 110 from position A to B is illustrated by an arrow in FIG. 1. The first measurement position A may according to this example be located on the lower arm whereas the second measurement position B may be located on the upper arm. As the upper arm and the lower arm are moveable relative to each other around the elbow joint 11, the range of motion of the elbow joint 11 may be estimated by measuring the relative angle between the upper arm and the lower arm when flexing the elbow joint 11. Alternatively, or additionally, the range of motion may be estimated by keeping the alignment guide 100 and sensor unit 110 in the first measurement position A and flex the arm around the elbow joint 11. By measuring the relative angle between the orientation of the sensor unit 110 in two different positions, that correspond to different degrees of motion, the range of motion may be determined.

Two further embodiments will be described. In a first embodiment a single sensor is used. In a second embodiment two sensors in communication with each other are used. It is also envisaged that three or more sensors can be used in some configurations. The sensor(s) may be similarly configured as the sensor or sensor unit discussed with reference to the embodiments of FIGS. 1 and 2.

First a description of an exemplary overall system and components used in the system is given. In accordance with the present invention there is provided a digital system for determining the motion and motion range of parts of the body. The digital system comprises one or more sensor units that can be fastened or held to the body. In the case of two sensor units a sensor can be positioned at both sides of an angle to be measured such as at both sides of a joint, In case of only one sensor the sensor is can first be placed at one position and then moved to another position in order to measure the angle between the two positions.

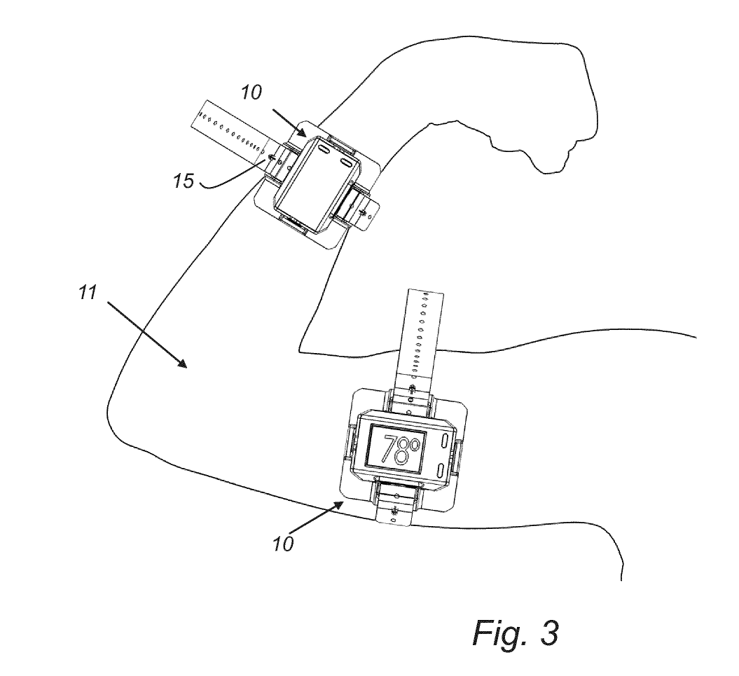

In FIG. 3, a view of a system for angular measurement of a body is depicted. The example in FIG. 3 comprises two sensors units 10. The sensor units 10 are placed at both sides of an elbow joint 11 and adapted to measure the angle of the elbow joint 11. The sensor units 10 can be fixed to the body by fasteners. In the embodiment in FIG. 3 the fasteners are straps. Other fasteners are of course also possible to use.

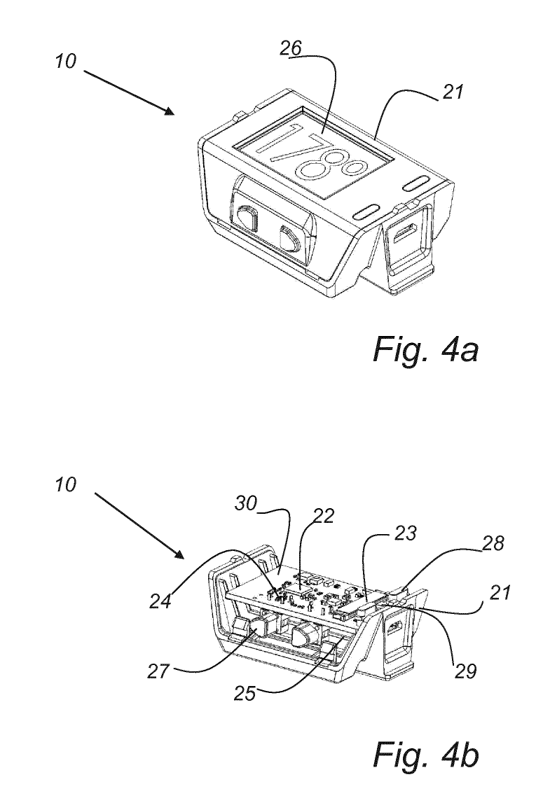

In FIGS. 4a and 4b, a view of a sensor unit 10 is depicted. The sensor unit may be similarly configured as the sensor units described with reference to any one of the previous figures. FIG. 4a depicts the unit assembled. FIG. 4b is identical with FIG. 4a but with a cover of the housing and display removed. A sensor unit 10 comprises an angle sensor for determining the angle of the unit. In accordance with one embodiment the angle sensor is implemented by an inertial measurement unit (IMU). The IMU can for example be a chip that is able to measure rotation in all three axes. This could be done using a 3 axis gyroscope. A 3-axis magnetometer and a 3 axis accelerometer can be added to further improve angle measurements. The units can further be adapted to communicate with other sensor units or a master unit or a display unit or a combination thereof via a wireless communication system.

In the exemplary sensor unit 10 depicted in FIGS. 4a and 4b, the sensor comprises a housing 21 in which components of the sensor unit are placed on a circuit board 30 and interconnected. The components are a processor unit 22, an angle sensor that can be in the form of an IMU (Inertial Measurement Unit) 23, a transceiver unit 24, a battery 25, a screen 26, input buttons 27, Light emitting diodes (LED) 28, a vibrator and a sound output device 29. The processor 22 controls the sensor unit 20. All computations can be performed by the processor 22. The IMU 23 can in one embodiment be a chip with a 3 dimensional gyroscope, a 3 dimensional accelerometer and a 3 dimensional magnetometer. The IMU can also host a processor provided to perform calculations for combining data from the sensors (gyroscope, magnetometer and accelerometer). The data can be represented as a quaternion and three scalars representing direction and acceleration along three axes in a three-dimensional coordination system, here termed x, y and z axis respectively. Other representations are possible.

The sensor unit 10 is provided with a transceiver 24 for communication with another sensor unit and/or with other units/devices.

Further, buttons 27 are provided to enable interaction with the sensor unit 10. Output data can be displayed on the screen 26 or sent wirelessly to a remote device (e.g. a smartphone or computer) and be displayed on such a remote device. Output from the sensor unit 10 can also be given via the LEDs 28, and/or the vibrator and/or the sound output device 29. The output can for example be in the form of light signals from the LEDs or a tone or similar from the sound output device 29.

### Single Sensor System

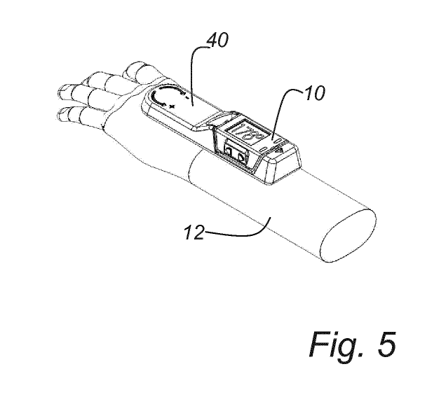

In case one single sensor is used to determine a body angle, the sensor unit 10 can be operated as follows. FIG. 5 depicts a one sensor configuration. The sensor unit 10 is aligned with a body part that is to be measured. For example if the movement of the elbow joint is to be measured, the sensor unit 10 can be aligned with the forearm 12. In order to facilitate the alignment the sensor unit 10 can be attached to a handle 40. For example projections on the sensor unit 10 can be clicked into corresponding recesses of the handle 40. The handle 40 is then aligned with the body part to be measured; in the given example the forearm 12. The handle 40 provides the advantage that it is easy to hold the sensor and also it is made easier to align the sensor unit correctly with the body part to be measured.

A single sensor measurement can typically be performed by a person other than the patient, such as a physical therapist. In use the sensor is aligned and started, for example by pressing one of the buttons 27 thereby fixing a first position, a start position. The body is then moved into another position and a stop command is given, for example by pressing the same or another button 27. The sensor unit 10 then calculates the angular difference between the start and stop positions and provides the resulting output via the screen or by sending it to an external device via the transceiver. The sensor unit 10 can also be configured to continuously output the angular difference as the body is moved to the stop position. Algorithms for calculating the angular difference are described in more detail below.

### Dual Sensor System

In case two sensors units 10 are used, the two sensors units 10 are positioned on the body at positions for which the relative motion is to be measured. In the dual sensor system configuration, two sensors units 10 of the kind described above can be used. The sensor units 10 can be attached to the body by a strap or a similar device. In FIG. 3 the sensor units 10 are strapped to the forearm and the upper arm to measure the elbow joint angle. The sensor units 10 can communicate with each other and calculate the relative angular difference between the sensor units. In order for the angular difference to be calculated correctly the two sensor units should have a common coordinate system. This can be achieved by positioning the two sensor units 10 in a known relative direction and calibrating the two sensors when placed in the known direction (for example when the two sensors are placed on top of each other). Other methods of calibrating the coordinate systems can be used such as placing the two sensors in a box with the two sensor units aligned in a known position. Further grooves can be provided on the sensor housings to facilitate alignment of the two sensors when performing calibration.

The system with two sensor units can be operated by (after calibration of the two sensor units and strapping them onto the body) either a physiotherapist or a patient who needs feedback on rehabilitation exercises starting the measurement by for example pressing a button 27. The relative angle between the sensor units 10 is then output by for example displaying the angle on the display of one or both of the sensor units 10 or transmitting the angular measurement to another unit/device such as a computer, a tablet or a smartphone and saving/displaying the data there. The starting and stopping of the measurement can be performed differently depending on the algorithm used to calculate the angular difference.

It is also possible to provide more than two sensor units 10 in the measuring system as described herein. This can for example be advantageous when complex anatomical movements/angles are to be measured such as body posture or measurement of the spine.

Algorithms for determining the angular difference are described in more detail below.

### Algorithms

To determine the angle between two sensor units (or two sensor unit positions) according to any one of the preceding embodiments and examples, a simple algorithm could be used. First the sensors may be calibrated by measuring and saving the direction during calibration, when the sensors are placed in a known relative direction relative each other. By applying the inverse of the calibration direction to all following directions a reference coordinate system is obtained without any rotation. All directions (angles) can for example be represented as quaternions. So in order to obtain the reference coordinate system the quaternion of the sensor unit at the time of calibration is saved as the start quaternion. Following quaternions are then multiplied with the inverse of this quaternion. This multiplication is performed for all subsequent quaternions in order for the two sensor units to measure in a common coordination system. During measurement two quaternions are received for each sample, one quaternion describing the direction for each sensor. The angular difference can then be obtained by multiplying the quaternion of one sensor with the inverse quaternion of the other sensor. The problem with such an algorithm is that all rotations are factored in and not only the rotation in the plane that is to be measured. To achieve a better result, such an algorithm is therefore advantageously modified. Below some examples are described where the main/pre-dominant rotation axis is first determined or approximated to enable elimination of at least most of the rotation components around all other axes. This will yield an angular measurement in the desired axis while disregarding rotations in other axes.

### Algorithm 1

In accordance with this algorithm the base vectors of the sensor units are utilized and it is required that a start position is indicated, i.e. when the examination is to be started. The algorithm is based on finding the pair of base vectors (one vector for each sensor) that changes most and is based on the assumption that the angle that is of interest is the angle that changes most. In other words the rotation axis is determined to be the cross product/vector product of the base vector pair that has changed the most. The angle is then determined as the arc cos of the scalar product of the base vectors that changes the most which is the same as the static angle between the sensors. Because the angle is determined as the scalar product of the base vector pair, the algorithm will not be sensitive to any rotation in axes that are parallel to the selected base vectors. The plane defined by the selected base vectors may be referred to as the main plane of rotation.

For example, if the sensor unit(s) are set up to measure the angle of the elbow joint the following scenario can take place. When the arm is lifted, the fore arm is at the same time twisted somewhat. The main rotation is for lifting the arm and the twist is an error component. In this example denote the vector along the arm as y. The base vector pair that is most changed is y for the two sensor units. The twisting around the y-axis is excluded by the scalar product operation.

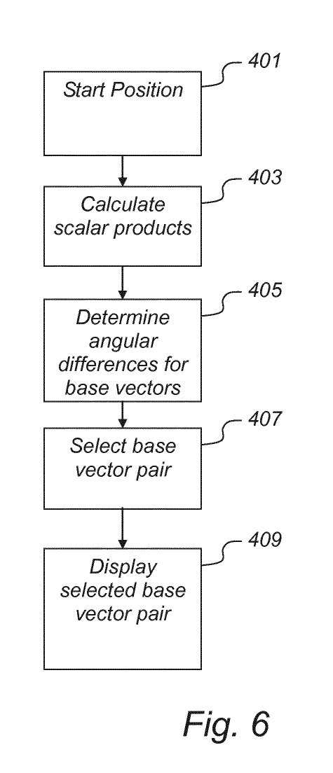

In FIG. 6, steps performed in such an algorithm are described. First in a step 401 a measurement starting position with an associated start angle is given. This can be performed by starting a new measurement. Next, in a step 403, the scalar product between all base vector pairs of the two sensors are calculated. In other words the scalar product between the x-axis of a first sensor unit and the x-axis of the second sensor unit is calculated and the scalar product between the x-axis of a first sensor unit and the y-axis of the second sensor unit is calculated etc. Hence, scalar products of all pairs of base vector pair are calculated. This corresponds to the starting angle for all base vector pairs. Then, in a step 405, the angle for all base vector pairs is computed minus the start angle for each base vector pair. Next, in a step 407, a base vector pair to be measured is determined. For example the first base vector pair that satisfies a particular condition or a set of conditions is determined to be the base vector pair to be used. The condition(s) can be any suitable condition such as the first base vector pair to exceed a particular angle, e.g. 10 degrees. Then, in a step 409, the angular difference of the base vector pair in step 407 is displayed. In some cases it may not be necessary to calculate the angle between all base vectors, it may be sufficient to calculate the angle between a few base vector pairs in order to determine the largest rotation.

In case only one sensor unit is used not only the start position needs to be entered but also the end position of the single sensor unit. The algorithm is then essentially the same and the scalar product for all base vector pairs for the start and end position is determined. The base vector pair giving the highest corresponding angle is determined as the measured angle and the corresponding angular measure is displayed when the end position is entered.

### Algorithm 2

In accordance with this, second, algorithm the plane of rotation is determined based on the motion of the sensor units. Because this typically requires that the sensor units follow the motion of the body it is more suited for a configuration where the sensor units are fixed to the body. Hence, because the one sensor system typically not fixes the sensor units to the body, this algorithm, algorithm 2, probably is more useful for a system with two (or more) sensor units.

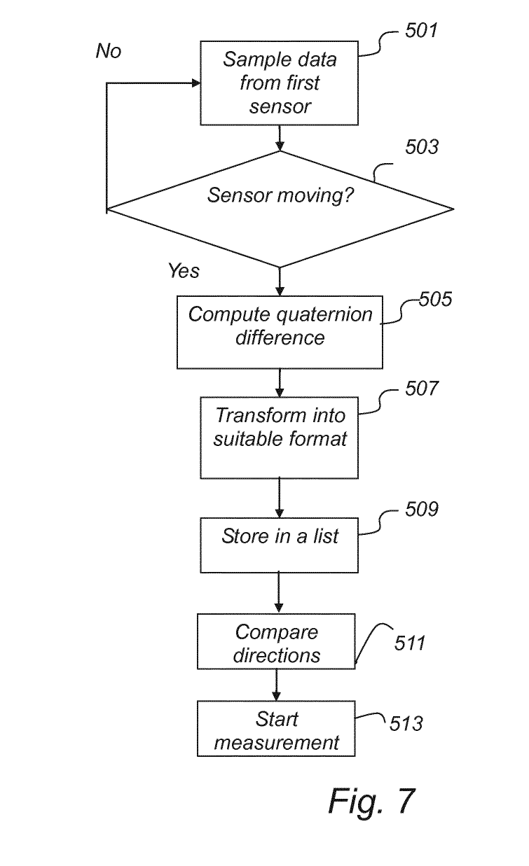

A main principle in accordance with the second algorithm is for the sensor unit(s) to sense in which plane it is rotating. The sensor unit that first detects a rotation in a plane is configured to rotate its coordination system to be aligned such that a base vector, e.g. the z-axis, is aligned with the axis of rotation. All other sensor units perform the corresponding transformation so that all coordination systems of all sensor units are aligned in accordance with the first sensor unit. When the angle between the sensor units then is computed, the angle is confined to the rotations around the axis perpendicular to the rotation plane. This will filter out any unwanted rotational components. The method above can be implemented as follows with reference to FIG. 7. First in a step 501 the quaternion and acceleration of a first sensor is sampled. Based on the sampled values detect if the first sensor is moving in a step 503. This can be performed in various ways. For example sampled acceleration data can be used or angular differences from previous samples can be used. Regardless of method if the sensor unit is not moving, the procedure of FIG. 7 returns to step 501 else the procedure proceeds to step 505.

In Step 505, the quaternion difference that describes the rotation between the current sample and the previous sample is then continuously computed. The quaternion difference is then transformed into an axis-angle or a similar representation in a step 507. The results in step 507 are stored in a list in a step 509. The list in step 509 will then comprise a number of directions and angles for each sample as compared to the previous sample. Next, in a step 511, all directions of the list formed in step 509 are compared to a reference direction to determine the variation of the direction. The reference direction can for example be the first direction or the average direction in the list. The variance of the sampled direction values are then used to determine if a movement is in one dominating direction. This is possible since a large variation (above a threshold value) indicates different directions in different planes (random movements) and a small variation (below a threshold value) indicates movement in only one plane. If the variation determined in step 511 is below a threshold value (the rotational axis is relatively fix), the reference axis used is saved and the coordinate systems of the sensors are transformed such that one axis (e.g. the z axis) is aligned with the reference axis. The corresponding transformation is done for all other sensor units. The measurement is then started in a step 513. The starting can be signaled for example via the LEDs or the sound output device. The angular difference between the sensor vectors aligned with the rotational axis (here the x or y axis) is computed and displayed as a static angle. If the variation is too large, e.g. above a threshold value, the sensor is not moving in one plane and the measurement is aborted. The reference direction is then reset and the procedure returns to step 501. This can be signaled for example via the LEDs or the sound output device. This method does not require any manual input of a start and stop event. In the above examples rotation is represented by quaternions. It is however to be understood that other methods of representing angles and direction can be used.

Using the methods and systems as described herein will provide many advantages compared to existing systems. For example, the systems are user friendly and also the output can be generated filtered from at least some of error components relating to other angles other than the anatomical angle to be measured, which improves the reliability of the measurement.

### Itemized List of Embodiments

1. A system for determining body motion, the system comprising at least one sensor unit (10), comprising an angle sensor (23), the system being adapted to determine the motion of a body part based on the relative angle of two positions of a sensor unit or the relative angle between two sensor units, the system being adapted to, for a particular angle to be measured, determine a plane in which a predominant rotation takes place and to output a measured angle around an axis perpendicular to the determined plane.
2. The system according to embodiment 1, wherein, when two angle sensor units (10) are used, the two sensor units (10) are calibrated to a common coordination system.
3. The system according to embodiment 1 or 2, wherein the angle sensor(s) (23) comprises an inertial measurement unit, IMU.
4. The system according to embodiment 3 wherein the IMU is adapted to output quaternion and direction samples.
5. The system according to any of embodiments 1-4, wherein the system is adapted to find the direction to be measured by determining the scalar product between two base vector pairs that is the largest as the measured direction.
6. The system according to any of embodiments 1-5 wherein the sensor units (10) comprises fastening means for attaching the sensor units (10) to a holder.
7. The system according to embodiment 6, wherein the holder is a handle (40).
8. The system according to embodiment 6, wherein the holder is a fastener (15).
9. The system according to any of embodiments 1-6 where at least three sensors are provided.

1-25. (canceled)

26. A system for determining a human or animal body part range of motion around a joint, said system comprising an alignment guide and a sensor unit, said alignment guide being adapted to be placed against the body part and to receive the sensor unit, wherein said sensor unit is adapted to estimate, in a main plane of rotation, the range of motion based on a relative angle between an orientation of the sensor unit in a first measurement position and an orientation of the sensor unit in a second measurement position.

27. The system according to claim 26, wherein the sensor unit is adapted to estimate the main plane of rotation based on the sensor unit's orientation in the first measurement position and the second measurement position, respectively.

28. The system according to claim 27, wherein the sensor unit is adapted to estimate the main plane of rotation by finding a maximum scalar product of a set of base vector pairs of the sensor unit in the first measurement position and the second measurement position, respectively.

29. The system according to claim 27, wherein the sensor unit is adapted to estimate the main plane of rotation by means of a spherical linear interpolation, SLERP, of quaternion representations of the orientation of the sensor unit in the first measurement position and the second measurement position, respectively.

30. The system according to claim 26, wherein the sensor unit comprises a gripping means having at least two pairs of substantially parallel surfaces adapted to be gripped between an index finger and a middle finger, the middle finger and the ring finger, or the ring finger and the little finger of a hand of a user during measurement.

31. The system according to claim 30, wherein the sensor unit further comprise an activation means (130) adapted to be pushed by a thumb of said hand so as to initiate a measurement of an orientation of the sensor unit.

32. The system according to claim 26, wherein the sensor unit comprises an inertial measurement unit, IMU.

33. The system according to claim 32, wherein the IMU is adapted to output quaternion and direction information.

34. A method for determining a human or animal body part range of motion around a joint by means of an alignment guide and a sensor unit being attached to the alignment guide, said method comprising the steps of:
placing the alignment guide against the body;
measuring an orientation of the sensor unit in a first measurement position;
measuring an orientation of the sensor unit in a second measurement position;
estimating, in a main plane of rotation, the range of motion based on a relative angle between the orientation of the sensor unit in the first measurement position and the orientation of the sensor unit in the second measurement position.

35. The method according to claim 34, comprising the step of moving the alignment between a first position on the body and a second position of the body part, said first position and second position corresponding to the first measurement position and the second measurement position, respectively.

36. The method according to claim 34, wherein the first measurement position and the second measurement position correspond to a same position on the body part.

37. The method according to claim 34, further comprising the step of estimating the main plane of rotation based on the sensor unit's orientation in the first measurement position and the second measurement position, respectively.

38. The method according to claim 34, further comprising the step of estimating the main plane of rotation based on the sensor unit's orientation in a plurality of points between the first measurement position and the second measurement position.

39. The method according to claim 34, wherein the steps of measuring the orientation of the sensor unit in the first measurement position and the second measurement position, respectively, is performed in response to receipt of an activation signal.

40. The method according to claim 39, wherein the activation signal is generated by pushing a button, tapping on the sensor unit or providing a voice command.

41. A method for determining a human or animal body part range of motion around a joint, comprising providing the system of claim 26, and applying the system so as to measure said range of motion.

42. The system according to claim 26, being adapted to, when placed against the body part, contact the body part in at least two distinct points of contact.
